(19) United States
(12) Patent Application Publication
(10) Pub. No.: US 2025/0344959 A1
(43) Pub. Date: Nov. 13, 2025

(54) DISPLAY DEVICE INCLUDING BIOMETRIC INFORMATION MEASUREMENT AREAS

(71) Applicant: SAMSUNG DISPLAY CO., LTD., YONGIN-SI (KR)

(72) Inventor: Chul KIM, Yongin-si (KR)

(21) Appl. No.: 18/968,536

(22) Filed: Dec. 4, 2024

(30) Foreign Application Priority Data
May 10, 2024 (KR) 10-2024-0061736

(51) Int. Cl. A61B 5/024; A61B 5/00

(52) U.S. Cl. CPC A61B 5/02433; A61B 5/6898

(57) ABSTRACT

A display device includes a display panel having a display area divided into a plurality of image display areas and a plurality of biometric information measurement areas, display pixels arranged in the plurality of image display areas and the plurality of biometric information measurement areas, light-sensing pixels arranged in the plurality of biometric information measurement areas, infrared light-emitting pixels arranged in the plurality of biometric information measurement areas, a display scan driver configured to drive the display pixels and the infrared light-emitting pixels to emit light, a light-sensing scan driver configured to drive the light-sensing pixels to sense light, and a main driver circuit configured to detect a user's pulse wave signals and measure biometric information using light-sensing signals received through the light-sensing pixels, wherein the plurality of biometric information measurement areas are formed in a long bar shape or a rectangular shape toward the main driver circuit.

### CROSS-REFERENCE TO RELATED APPLICATION

This application claims priority under 35 U.S.C. 119 from Korean Patent Application No. 10-2024-0061736, filed on May 10, 2024, in the Korean Intellectual Property Office, the disclosure of which is herein incorporated by reference in its entirety.

### BACKGROUND

### 1. Technical Field

The present disclosure relates to a display device, and more particularly to a display device including biometric information measurement areas.

### 2. Discussion of Related Art

As information-oriented societies evolve, various demands for display devices are being made in connection with the manner in which images may be displayed. Display devices are being employed by a variety of electronic devices such as smart phones, digital cameras, laptop computers, table PCs, navigation devices, and smart televisions. Portable display devices such as smartphones and tablet PCs may be equipped with a variety of features including image capturing, fingerprint recognition, or face recognition.

Recently, efforts have been made to combine portable display devices such as smartphones or tablet PCs with portable biometric detection devices. These biometric devices may measure various biometric information such as heart rate, heart rate variability, respiration, cardiovascular disease and oxygen saturation in addition to blood pressure.

### SUMMARY

Aspects of the present disclosure provide a device that can detect photoplethysmography signals and measure a user's biometric information such as blood pressure using a back type display panel having a driver circuit disposed at a side.

Aspects of the present disclosure may also provide a display device in which display pixels and light-sensing pixels may be disposed in a plurality of biometric information measurement areas in an image display area of the display panel, and biometric information may be detected using pulse wave signals detected through the plurality of biometric information measurement areas.

It should be noted that objects of the present disclosure are not limited to the above-mentioned object; and other objects of the present disclosure will be apparent to those skilled in the art from the present description.

According to an embodiment of the disclosure, a display device includes a display panel having a display area divided into a plurality of image display areas and a plurality of biometric information measurement areas, a plurality of display pixels arranged in the plurality of image display areas and the plurality of biometric information measurement areas, a plurality of light-sensing pixels arranged in the plurality of biometric information measurement areas, a plurality of infrared light-emitting pixels arranged in the plurality of biometric information measurement areas, a display scan driver configured to drive the plurality of display pixels and the plurality of infrared light-emitting pixels to emit light, a light-sensing scan driver configured to drive the plurality of light-sensing pixels to sense light, and a main driver circuit configured to detect a pulse wave signal and measure biometric information using light-sensing signals received from the plurality of light-sensing pixels, wherein the plurality of biometric information measurement areas are disposed in parallel rectangular shapes.

According to an embodiment of the disclosure, a display device includes a display panel having a display area divided into a plurality of image display areas and a plurality of biometric information measurement areas, a plurality of display pixels arranged in the plurality of image display areas and the plurality of biometric information measurement areas, a plurality of light-sensing pixels arranged in the plurality of biometric information measurement areas, a plurality of infrared light-emitting pixels arranged in the plurality of biometric information measurement areas, a display scan driver configured to drive the plurality of display pixels and the plurality of infrared light-emitting pixels to emit light, a light-sensing scan driver configured to drive the plurality of light-sensing pixels to sense light, and a main driver circuit configured to detect a user's pulse wave signals and measure biometric information using light-sensing signals received from the plurality of light-sensing pixels, wherein the plurality of image display areas and the plurality of biometric information measurement areas are arranged in the display area alternately in a first direction, and are arranged in parallel in the display area in a second direction intersecting the first direction.

According to an embodiment of the disclosure, a display device includes a display panel display panel having a display area divided into a plurality of image display areas and a plurality of biometric information measurement areas; a plurality of display pixels arranged in the plurality of image display areas and the plurality of biometric information measurement areas; a plurality of light-sensing pixels arranged in the plurality of biometric information measurement areas; a plurality of infrared light-emitting pixels arranged in the plurality of biometric information measurement areas; a display scan driver configured to drive the plurality of display pixels and the plurality of infrared light-emitting pixels to emit light; a light-sensing scan driver configured to drive the plurality of light-sensing pixels to sense light; and a main driver circuit configured to receive light-sensing signals from the plurality of light-sensing pixels, wherein a first biometric information measurement area among the plurality of biometric information measurement areas is extended in a length direction of the display area between a first image display area and a central image display area, which are extended in a length direction of the display panel, and wherein a second biometric information measurement area among the plurality of biometric information measurement areas is extended in the length direction of the display area between the central image display area and a second image display area, which are extended in the length direction of the display panel, wherein the first image display area and the second image display area are disposed on opposite sides of the central image display area.

According to an embodiment of the present disclosure, it may be possible to detect photoplethysmography signals and measure a user's biometric information such as blood pressure using a back type display panel having a driver circuit disposed at a side in a display device. As a result, the design structure of the display panel can be simplified and fabrication costs can be reduced.

In addition, according to an embodiment of the present disclosure, light-sensing pixels may be disposed together with display pixels in a plurality of biometric information measurement areas of a display area in a display device, and biometric information can be detected using pulse wave signals detected through the plurality of biometric information measurement areas. As a result, the biometric information such as blood pressure, heart rate, heart rate variability, respiratory rate and oxygen saturation can be measured more accurately.

It should be noted that effects of the present disclosure are not limited to those described above and other effects of the present disclosure will be apparent to those skilled in the art from the present description.

### BRIEF DESCRIPTION OF THE DRAWINGS

The above and other aspects and features of the present disclosure will become more apparent by describing in detail embodiments thereof with reference to the attached drawings, in which:

FIG. 1 is a perspective view of a display device according to an embodiment of the present disclosure.

FIG. 2 is a plan view showing an arrangement structure of a display panel and a main driver circuit shown in FIG. 1 according to an embodiment.

FIG. 3 is a side view specifically showing a configuration of the display device shown in FIG. 1 and FIG. 2.

FIG. 4 is a side view specifically showing a configuration of the display device shown in FIG. 1 and FIG. 2 according to another embodiment.

FIG. 5 is a view showing an example of a layout of the display panel shown in FIGS. 1 to 4.

FIG. 6 is a layout view showing arrangement structure of pixels in an image display area of a display area according to an embodiment.

FIG. 7 is a circuit diagram showing first and second display pixels arranged in the image display area of FIG. 6.

FIG. 8 is a layout view showing arrangement structure of pixels in a biometric information measurement area of a display area according to an embodiment.

FIG. 9 is a circuit diagram showing a display pixel and a light-sensing pixel disposed in the biometric information measurement area of FIG. 8.

FIG. 10 is a view showing a method of measuring pulse wave signals at a plurality of touch locations through a plurality of biometric information measurement areas.

FIG. 11 is a plan view showing an arrangement structure of a display panel and a main driver circuit shown in FIG. 1 according to an embodiment.

FIG. 12 is a plan view showing an arrangement structure of a display panel and a main driver circuit shown in FIG. 1 according to an embodiment.

FIG. 13 is a view showing a method of measuring pulse wave signals at a plurality of touch locations through a plurality of biometric information measurement areas.

FIG. 14 is a waveform diagram showing a pulse wave signal of a finger detected in the first biometric information measurement area.

FIG. 15 is a waveform diagram showing a pulse wave signal of a finger detected in the second biometric information measurement area.

FIG. 16 is a diagram for illustrating a method of calculating blood pressure information using a machine learning algorithm according to an embodiment.

FIG. 17 is a graph for illustrating a method of calculating information about heart rate and respiration among biometric information according to an embodiment.

FIG. 18 is a graph for illustrating a method for calculating information about blood vessel elasticity among biometric information according to an embodiment.

FIG. 19 is a graph for illustrating a method for calculating information about cardiovascular disease among biometric information according to an embodiment.

FIG. 20 is a graph for illustrating a method for calculating information about oxygen saturation among biometric information according to an embodiment.

FIG. 21 is a diagram showing the results of biometric information measurement using the display device according to the embodiment of the present disclosure.

### DETAILED DESCRIPTION OF THE EMBODIMENTS

The present disclosure will now be described more fully hereinafter with reference to the accompanying drawings, in which preferred embodiments of the disclosure are shown. Aspects of the present disclosure may, however, be embodied in different forms and should not be construed as limited to embodiments set forth herein. Rather, embodiments are provided so that this disclosure will be thorough and complete, and will filly convey the scope of the disclosure to those skilled in the art.

It will also be understood that when a layer is referred to as being “on” another layer or substrate, the layer can be directly on the other layer or substrate, or intervening layers may also be present. In contrast, when an element is referred to as being “directly on” another element, there may be no intervening elements present. The same reference numbers indicate the same components throughout the specification.

It will be understood that, although the terms “first,” “second,” etc. may be used herein to describe various elements, these elements should not be limited by these terms. These terms are only used to distinguish elements. For example, a first element discussed herein could be termed a second element without departing from the teachings of the present disclosure. Similarly, the second element could also be termed the first element.

Features of different embodiments of the present disclosure may be implemented individually or combined with each other, in part or in whole. Embodiments may be implemented independently of each other or may be implemented together in an association.

Hereinafter, embodiments of the present disclosure will be described with reference to the accompanying drawings.

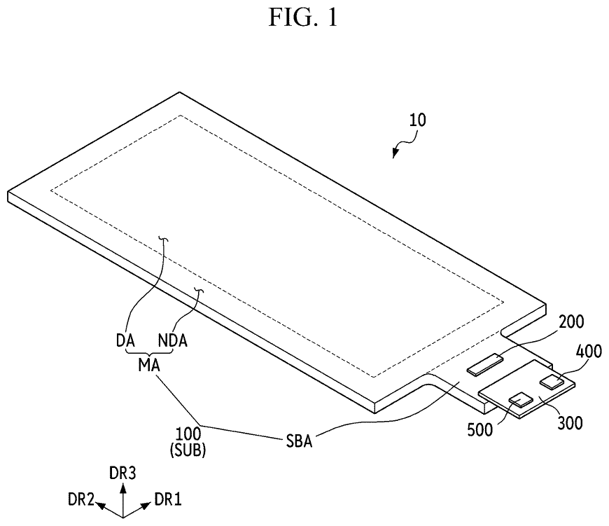

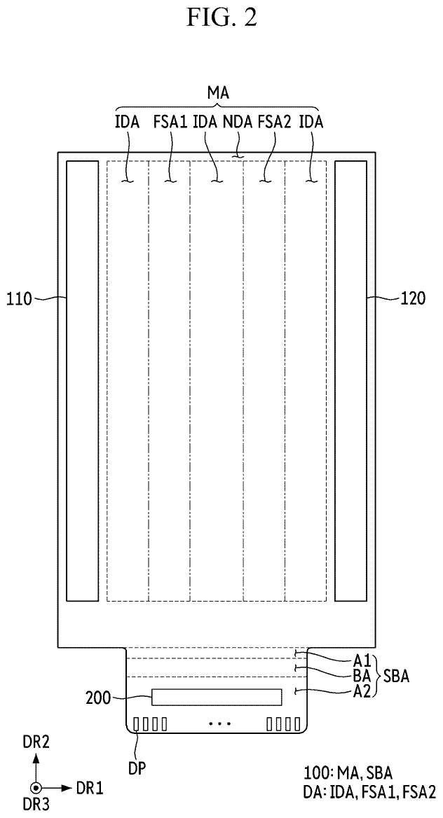

FIG. 1 is a perspective view showing a display device according to an embodiment of the present disclosure. FIG. 2 is a plan view showing an arrangement structure of a display panel and a main driver circuit shown in FIG. 1 according to an embodiment.

Referring to FIG. 1 and FIG. 2, a display device 10 according to an embodiment of the present disclosure may be employed by portable electronic devices such as a mobile phone, a smart phone, a tablet PC, a mobile communications terminal, an electronic notebook, an electronic book, a portable multimedia player (PMP), a navigation device, or a ultra mobile PC (UMPC). In addition, the display device 10 according to an embodiment of the present disclosure may be used as a display unit of a television, a laptop computer, a monitor, an electronic billboard, or an Internet of Things (IoT) device. Alternatively, the display device 10 according to an embodiment of the present disclosure may be applied to wearable devices such as a smart watch, a watch phone, a glasses-type display, or a head-mounted display (HMD) device. In addition, the display device 10 according to an embodiment may be used as a center information display (CID) disposed at the instrument cluster, the center fascia or the dashboard of a vehicle, as a room mirror display on the behalf of the side mirrors of a vehicle, or as a display placed on the back of each of the front seats that is an entertainment system for passengers at the rear seats of a vehicle.

The display device 10 may be a light-emitting display device such as an organic light-emitting display device using organic light-emitting diodes, an inorganic light-emitting display device including an inorganic semiconductor, or a micro light-emitting display device using micro or nano light-emitting diodes (micro LEDs or nano LEDs). In the present description, an organic light-emitting display device is described as an example of the display device 10. It is, however, to be understood that the present disclosure is not limited thereto.

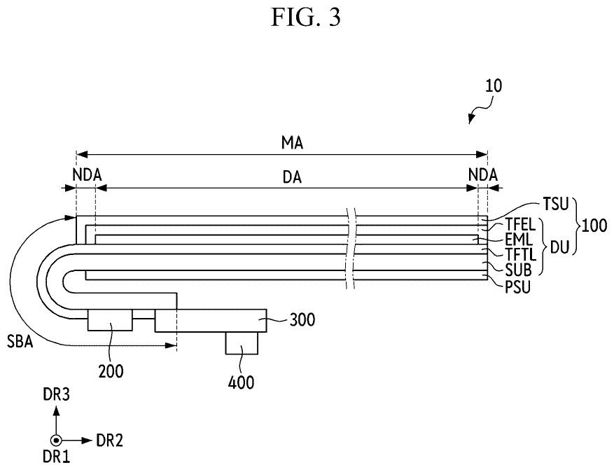

FIG. 3 is a side view specifically showing a configuration of the display device shown in FIG. 1 and FIG. 2.

Referring to FIG. 2 and FIG. 3, the display device 10 may include a display panel 100, a main driver circuit 200, a touch sensing unit TSU, a pressure sensing unit PSU, a circuit board 300, and a touch driver circuit 400.

In the present description, elements, components, or features may be described in the context of a first direction DR1, a second direction DR2, and a third direction DR3. The second direction DR2 may intersect the first direction DR1. The second direction DR2 may be perpendicular to the first direction DR1. The first direction DR1 and the second direction DR2 may form a horizontal plane. The third direction DR3 may be a vertical direction intersecting both the first direction DR1 and the second direction DR2.

The display panel 100 may be formed in a rectangular shape. For example, the display panel 100 may having shorter sides in the first direction DR1 and longer sides in the second direction DR2 intersecting the first direction DR1 when viewed from the top. Each of the corners where two sides meet may be formed at a right angle or may be rounded with a predetermined curvature. The shape of the display panel 100 when viewed from the top is not limited to a quadrangular shape, but may be formed in a different polygonal shape, a circular shape, or an elliptical shape. The display panel 100 may be formed substantially flat, e.g., within manufacturing tolerances, but the present disclosure is not limited thereto. For example, the display panel 100 may be formed at left and right ends, and may include a curved portion having a constant curvature or a varying curvature. In addition, the display panel 100 may be flexible so that the display panel 100 may be curved, bent, folded, or rolled.

A substrate SUB of the display panel 100 may include a main area MA and a subsidiary area SBA.

The main area MA may include a display area DA where images may be displayed, and a non-display area NDA at a side of the display area DA.

The non-display area NDA may be disposed adjacent to the display area DA. The non-display area NDA may be located on an outer side of the display area DA. The non-display area NDA may surround the display area DA. The non-display area NDA may be defined as the border of the display panel 100.

Referring to FIG. 2 and FIG. 3, the subsidiary area SBA may protrude from a side of the main area MA in the second direction DR2. The length of the subsidiary area SBA in the second direction DR2 may be different than the length of the main area MA in the second direction DR2. For example, the length of the subsidiary area SBA in the second direction DR2 may be smaller than the length of the main area MA in the second direction DR2. The length of the subsidiary area SBA in the first direction DR1 may be substantially less than the length of the main area MA in the first direction DR1 or may be substantially equal to it. It is, however, to be understood that the present disclosure is not limited thereto.

The subsidiary area SBA may include a first area A1, a second area A2, and a bending area BA.

The first area A1 may protrude from a side of the main area MA in the second direction DR2. A side of the first area A1 may be in contact with the non-display area NDA of the main area MA, and an opposite side of the first area A1 may be in contact with the bending area BA.

Pads DP and the main driver circuit 200 may be disposed in the second area A2. The main driver circuit 200 may be attached to driving pads of the second area A2 using a conductive adhesive member such as an anisotropic conductive film. The circuit board 300 may be attached to the pads DP of the second area A2 using a conductive adhesive member. A side of the second area A2 may be in contact with the bending area BA.

The bending area BA is a part of the display panel 100 that is bendable. When the bending area BA is bent, the second area A2 may be disposed under the first area A1 and under the main area MA. The bending area BA may be disposed between the first area A1 and the second area A2. A side of the bending area BA may be in contact with the first area A1, and the opposite side of the bending area BA may be in contact with the second area A2.

As shown in FIG. 3, the subsidiary area SBA may be bent so that it is located under the main area MA. The subsidiary area SBA may overlap with the main area MA in the thickness direction DR3.

The circuit board 300 may be attached to an end of the subsidiary area SBA. In other words, the circuit board 300 may be electrically connected to the subsidiary area SBA on a side of the display panel 100, so that it may be disposed in the display panel 100 as a single back type. The circuit board 300 may be electrically connected to the main driver circuit 200 and the touch driver circuit 400 on a side of the display panel 100.

The display panel 100 and the main driver circuit 200 may receive digital video data, timing signals, and driving voltages through the circuit board 300. The circuit board 300 may be a flexible printed circuit board, a printed circuit board, or a flexible film such as a chip-on film.

The display area DA may include display pixels configured to display images, and light-sensing pixels configured to sense light. This light may be reflected off a part of a user's body, such as a finger. In addition, the display area DA may further include infrared light-emitting pixels configured to emit infrared light.

Referring to FIG. 2 and FIG. 3, the display area DA may occupy most of the main area MA. The display area DA may be disposed at the center of the main area MR.

The display area DA may be divided into an image display area IDA in which display pixels may be disposed without light-sensing pixels, and biometric information measurement areas FSA1 and FSA2 in which light-sensing pixels as well as display pixels may be disposed. In other words, the light-sensing pixels which are formed to sense light incident on, or reflected from the front surface may be disposed together with the display pixels in a predetermined part of the entire display area DA of the display panel 100. For example, in an embodiment, the light-sensing pixels may be disposed together with the display pixels in the information measurement areas FSA1 and FSA2. In addition, the biometric information measurement areas FSA1 and FSA2 may further include infrared light-emitting pixels in addition to the display pixels and the light-sensing pixels.

Each of the biometric information measurement areas FSA1 and FSA2 may have a rectangular shape with longer side in the length direction of the display panel 100, e.g., the second direction DR2 and shorter sides in the width direction of the display panel 100. For example, the biometric information measurement areas FSA1 and FSA2 may have a bar shape.

The biometric information measurement areas FSA1 and FSA2 may be formed parallel to the length direction of the display panel 100 where the main driver circuit 200 is disposed, e.g., in the second direction DR2 and may be equally spaced apart from each other between adjacent image display areas IDA.

For example, among the plurality of biometric information measurement areas FSA1 and FSA2, the first biometric information measurement area FSA1 may extend in the length direction between an image display area IDA on a side and an image display area IDA in the center extended in the length of the display panel 100. For example, the plurality of biometric information measurement areas FSA1 and FSA2 may extend in parallel to each other. For example, the plurality of biometric information measurement areas FSA1 and FSA2 may be disposed as parallel rectangular shapes. Among the plurality of biometric information measurement areas FSA1 and FSA2, the second biometric information measurement area FSA2 may be extended in the length direction between the image display area IDA in the center and an image display area IDA on the opposite side extended in the length of the display panel 100. For example, the plurality of biometric information measurement areas FSA1 and FSA2 may be disposed on opposite sides of the image display area IDA.

The display area DA may be divided into the image display area IDA on a side, the first biometric information measurement area FSA1, the central image display area IDA, the second biometric information measurement area FSA2 and the image display area IDA on the opposite side in the width direction. The image display area IDA on a side, the first biometric information measurement area FSA1, the central image display area IDA, the second biometric information measurement area FSA2 and the image display area IDA on the opposite side may be extended in the length direction of the display area DA and parallel to one another. For example, the plurality of biometric information measurement areas FSA1 and FSA2 may be equally spaced apart from each other between the image display areas IDA.

The main driver circuit 200 may be disposed at a side of the display area DA, which may be the side of the display area DA in the length direction, and may be disposed on the circuit board 300 electrically connected to the side of the display area DA. For example, the main driver circuit 200 may be disposed at an end of the display area DA, which may be the end of the display area DA in the length direction.

Alternatively, the biometric information measurement areas FSA1 and FSA2 may be formed perpendicular to the length direction of the display panel 100, e.g., in the first direction DR1 and may be equally spaced apart from each other between adjacent image display areas IDA. For example, the display areas IDA may extend in the first direction DR1. In this case, the main driver circuit 200 may be disposed at a side of the display area DA, which is the side of the display area DA in a width direction, and may be disposed on the circuit board 300 electrically connected to the side of the display area DA.

The main driver circuit 200 may generate electric signals for driving the display panel 100. These electric signals may include, for example, control signals and data voltages. Each of the main driver circuit 200 and the touch driver circuit 400 may be implemented as an integrated circuit (IC). Each of the main driver circuit 200 and the touch driver circuit 400 may be attached to the display panel 100 or the circuit board 300 by a chip-on-glass (COG) technique, a chip-on-plastic (COP) technique, or an ultrasonic bonding. It is, however, to be understood that the present disclosure is not limited thereto. For example, the main driver circuit 200 and the touch driver circuit 400 may be attached on the circuit board 300 by a chip-on-film (COF) technique.

The touch sensing unit TSU that senses a part of a user's body such as a finger and an electronic pen may be formed or disposed on the front surface of the display panel 100. The touch sensing unit TSU may include a plurality of touch electrodes to sense a user's touch by capacitive sensing.

The touch sensing unit TSU may include a plurality of touch electrodes arranged such that they cross each other in the first and second directions DR1 and DR2. Specifically, the plurality of touch electrodes may include a plurality of driving electrodes arranged in parallel and spaced apart from one another in the first direction DR1, and a plurality of sensing electrodes arranged in parallel and spaced apart from one another in the second direction DR2 such that they may cross the driving electrodes with an organic material layer or an inorganic material layer therebetween. The driving electrodes and the sensing electrodes may extend in a wiring area between display pixels and the light-sensing pixels so that they do not overlap with the display pixels SP or the light-sensing pixels arranged in the display area DA. Such driving electrodes and sensing electrodes form mutual capacitance, and transmit touch sensing signals that may change according to a user's touch to the touch driver circuit 400.

The touch driver circuit 400 may supply touch driving signals to the plurality of driving electrodes, and may receive touch sensing signals from the plurality of sensing electrodes RE. Then, the touch driver circuit 400 senses changes in mutual capacitance between the driving electrodes and the sensing electrodes based on changes in the magnitude of the touch sensing signals. The touch driver circuit 400 generates touch data based on the changes in mutual capacitance between driving electrodes and sensing electrodes and identifies positions where the touches have been sensed. The touch driver circuit 400 generates touch data including a plurality of touch positions and a plurality of touch position coordinates based on changes in the capacitance of the touch nodes in the touch sensing unit TSU. Accordingly, the touch driver circuit 400 may provide to the main driver circuit 200 the coordinate data of the plurality of touch positions where the touches have been sensed.

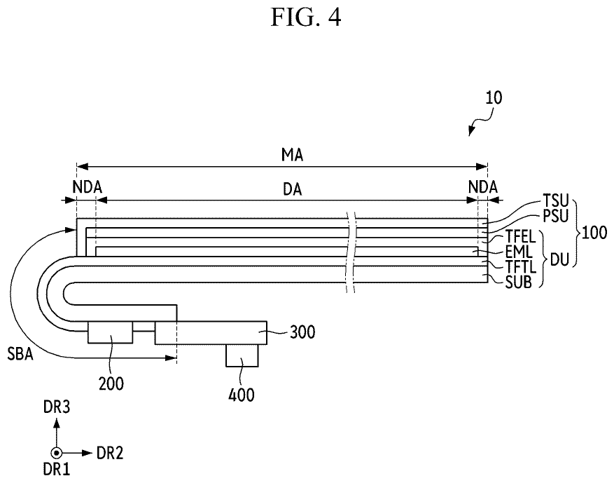

FIG. 4 is a side view specifically showing a configuration of the display device shown in FIG. 1 and FIG. 2 according to another embodiment.

The pressure sensing unit PSU that senses pressure applied by a part of a user's body such as a finger may be disposed or formed on the front surface of the display panel 100, e.g., on the surface between the display panel 100 and the touch sensing unit TSU. The pressure sensing unit PSU may be formed on the rear surface of the substrate SUB.

The pressure sensing unit PSU is required to detect absolute measurements associated with blood pressure, but may not be required to detect relative measurements associated with blood pressure. Therefore, the pressure sensing unit PSU may not be formed as shown in FIG. 3.

When the pressure sensing unit PSU is formed, the pressure sensing unit PSU may be formed as a transparent sheet in which transparent electrodes are arranged in vertical and horizontal directions, and may be disposed on the front surface of the main area MA. Alternatively, the pressure sensing unit PSU may be disposed or formed inside or on the front surface of the display panel 100.

The pressure sensing unit TSU may include a plurality of pressure sensing electrodes arranged such that they cross each other in the first and second directions DR1 and DR2. The plurality of pressure sensing electrodes may include a plurality of lower electrodes arranged in parallel and spaced apart from one another in the first direction DR1, and a plurality of upper electrodes arranged in parallel and spaced apart from one another in the second direction DR2, the upper electrodes crossing the lower electrodes with a transparent inorganic (or organic) material layer therebetween. Such lower electrodes and upper electrodes form mutual capacitance with a transparent inorganic (or organic) material layer, and transmit pressure-sensing signals that may change according to a user's touch to the touch driver circuit 400.

When the pressure sensing unit PSU is disposed on the front surface of the display panel 100, the pressure sensing electrodes of the pressure sensing unit PSU, i.e., a plurality of lower electrodes and upper electrodes may extend in the wiring area between the display pixels and the light-sensing pixels, so that they do not overlap with the display pixels and light-sensing pixels arranged in the display area DA. The touch driver circuit 400 may provide a reference voltage to the lower electrodes of the pressure sensing unit PSU, may receive pressure-sensing signals from the upper electrodes, and may sense changes in self-capacitance of the pressurized areas through the pressure-sensing signals. Accordingly, the touch driver circuit 400 may generate pressure data according to changes in the self-capacitance and sense coordinate data of the position where the pressure is sensed to provide them to the main driver circuit 200. The pressure sensing unit PSU may have various other structures in addition to the structure using the pressure sensing electrodes, and is not limited to that shown in FIG. 3 and FIG. 4.

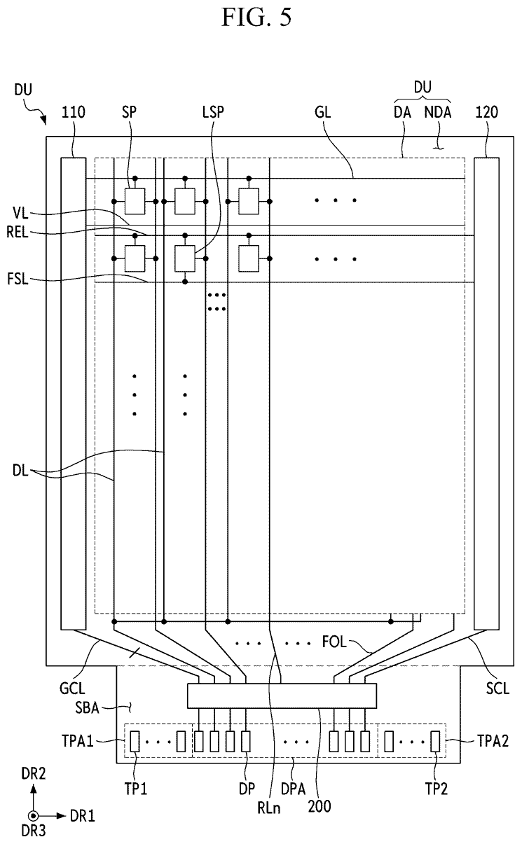

FIG. 5 is a view showing an example of a layout of the display panel shown in FIGS. 1 to 4. Specifically, FIG. 5 is a layout view showing the display area DA and the non-display area NDA of the display module DU before the touch sensing unit TSU is formed.

Referring to FIG. 4 and FIG. 5, in the display panel 100 of the display device 10 according to an embodiment, a display scan driver 110, a light-sensing scan driver 120 and the main driver circuit 200 may be disposed. In addition, the touch driver circuit 400 and a power supply unit (not shown) may be disposed on the circuit board 300 connected to the display panel 100. The main driver circuit 200 and the touch driver circuit 400 may be implemented as an integrated one-chip and may be mounted on the display panel 100 or the circuit board 300. In the present description, the main driver circuit 200 and the touch driver circuit 400 may be formed as different integrated circuits for convenience of illustration.

Referring to FIG. 5, the display panel 100 may include display pixels SP, light-sensing pixels LSP, display scan lines GL, emission control lines VL, data lines DL, sense scan lines FSL, sense reset lines REL, and light-sensing lines RLn disposed in the display area DA. The display scan driver 110 and the light-sensing scan driver 120 may be disposed in the non-display area NDA.

The display scan lines GL sequentially supply display scan signals applied for each horizontal line from the display scan driver 110 to the display pixels SP and the light-sensing pixels LSP for each horizontal line. The display scan lines GL may extend in the first direction DR1 and may be spaced apart from one another in the second direction DR2 intersecting the first direction DR1.

The emission control lines VL sequentially supply emission control signals applied for each horizontal line from the display scan driver 110 to the display pixels SP and the light-sensing pixels LSP for each horizontal line. The emission control lines VL may extend in the first direction DR1 in parallel with the display scan lines GL and may be spaced apart from one another in the second direction DR2 intersecting the first direction DR1.

The data lines DL may provide the data voltage received from the main driver circuit 200 to the plurality of display pixels SP. The plurality of data lines DL may extend in the second direction DR2 and may be spaced apart from one another in the first direction DR1.

The light-sensing scan lines FSL sequentially provide sense scan signals applied from the light-sensing scan driver 120 for each horizontal line to a plurality of light-sensing pixels LSP. The plurality of light-sensing scan lines FSL may extend in the first direction DR1 and may be spaced apart from one another in the second direction DR2 intersecting the first direction DR1.

The sense reset lines REL sequentially supply sense reset signals applied for each horizontal line from the light-sensing scan driver 120 to the light-sensing pixels LSP for each horizontal line. The sense reset lines REL may extend in the first direction DR1 in parallel with the light-sensing scan lines FSL and may be spaced apart from one another in the second direction DR2 intersecting the first direction DR1.

The light-sensing lines RLn may be connected between the respective light-sensing pixels LSP and the main driver circuit 200 to provide the light-sensing signals output from the respective light-sensing pixels LSP to the main driver circuit 200. The light-sensing lines RLn may be arranged to extend in the second direction DR2 according to the arrangement direction of the main driver circuit 200 and may be spaced apart from one another in the first direction DR1. The light-sensing lines RLn may be formed and disposed in the biometric information measurement areas FSA1 and FSA2 where the light-sensing pixels LSP are formed.

The non-display area NDA may surround the display area DA. This non-display area NDA may include the display scan driver 110, the light-sensing scan driver 120, fan-out lines FOL, gate control lines GCL, and sense control lines SCL.

In each of the biometric information measurement areas FSA1 and FSA2, a plurality of display pixels SP and a light-sensing pixel LSP may form a first unit pixel. The first unit pixels may be arranged in a matrix pattern along the first direction DR1 and the second direction DR2. When at least one infrared light-emitting pixel is additionally disposed in each of the biometric information measurement areas FSA1 and FSA2, a plurality of display pixels SP and an infrared light-emitting pixel may form a second unit pixel. Second unit pixels may be arranged in a matrix pattern in the display area DA such that they alternate with the first unit pixels.

For example, three display pixels SP that respectively display red, green, and blue lights and a light-sensing pixel LSP may form a single first unit pixel. In addition, three display pixels SP that respectively display red, green and blue lights, and an infrared light-emitting pixel may form a single second unit pixel. The first unit pixels and the second unit pixels may be alternately arranged in horizontal or vertical stripes to form a matrix pattern. Alternatively, the first unit pixels and the second unit pixels may be alternately arranged in a zigzag pattern when viewed from the top, and may be arranged in a matrix pattern in a diagonal direction.

Each of the red, green and blue display pixels SP and the infrared light-emitting pixels may be connected to a display scan line of the display scan lines GL and an emission control line of the emission control lines VL. During an image display period, red, green and blue display pixels SP may receive the data voltage from the data line DL in response to the display scan signal from the display scan line GL and the emission control signal from the emission control line VL, and may emit light by providing a driving current to the light-emitting element according to the data voltage. During a biometric information measurement period such as blood pressure, heart rate, oxygen saturation and vascular elasticity, the display pixels SP representing at least one color among red, green and blue display pixels SP may selectively receive a data voltage for emission along with a display scan signal and an emission control signal to emit light. In addition, during the biometric information measurement period such as blood pressure and heart rate, the infrared light-emitting pixels may selectively receive a data voltage for light emission along with a display scan signal and an emission control signal to display infrared light.

The light-sensing pixels LSP may be alternately arranged with the red, green and blue display pixels SP in the vertical or horizontal direction. Each of the light-sensing pixels LSP may be connected to a light-sensing scan line of the light-sensing scan lines FSL, a sense reset line of the sense reset lines REL, and a light-sensing line of the light-sensing lines RLn. During the biometric information measurement period such as blood pressure, respiratory rate, oxygen saturation and cardiovascular disease, the light-sensing pixels LSP may be reset in response to a sense reset signal from sense reset lines REL, and may generate a light-sensing signal in proportional to the amount of reflected light incident from the front side. In addition, each of the light-sensing pixels LSP may transmit the light-sensing signals to the light-sensing lines RLn in response to the sense scan signal from the light-sensing scan lines FSL.

Alternatively, the light-sensing pixels LSP may be connected to the display scan lines GL, respectively, for each horizontal line. Each of the light-sensing pixels LSP may generate a light-sensing signal proportional to the amount of reflected light incident from the front side, and may output the light-sensing signal to the light-sensing line RLn in response to a display scan signal input through the display scan line GL.

The display scan driver 110 may be disposed in the non-display area NDA. Although the display scan driver 110 is disposed on a side (e.g., the left side) of the display panel 100 in the drawings, embodiments of the present disclosure are not limited to that shown in the drawings. For example, the display scan driver 110 may be disposed on sides (e.g., left and right sides) of the display panel 100.

The display scan driver 110 may be electrically connected to the main driver circuit 200 through gate control lines GCL. The display scan driver 110 receives a scan control signal from the main driver circuit 200, sequentially generates display scan signals for each horizontal line driving period in response to the scan control signal, and sequentially supplies them to the display scan lines GL. In addition, the display scan driver 110 may sequentially generate emission control signals according to the scan control signal from the main driver circuit 200 and may sequentially supply them to the emission control lines VL.

The gate control line GCL may extend from the main driver circuit 200 to the display scan driver 110 depending on the position of the display scan driver 110. The gate control line GCL may supply the scan control signal received from the main driver circuit 200 to the display scan driver 110.

The light-sensing scan driver 120 may be disposed at a position in the non-display area NDA which is different from the position of the display scan driver 110. Although the light-sensing scan driver 120 is disposed on the other side (e.g., right side) of the display panel 100 in the example shown in FIG. 5, the present disclosure is not limited thereto. The light-sensing scan driver 120 may be electrically connected to the main driver circuit 200 through light-sensing control lines SCL. The light-sensing scan driver 120 receives a light-sensing control signal from the main driver circuit 200, and sequentially generates reset control signals and sense scan signals for each horizontal line driving period according to the light-sensing control signal. Then, the light-sensing scan driver 120 may sequentially provide the sequentially generated reset control signals to the sense reset lines REL. In addition, the light-sensing scan driver 120 may sequentially generate the sense scan signals in response to the light-sensing control signal from the main driver circuit 200 and sequentially provide them to the sense scan lines FSL.

The light-sensing control line SCL may extend from the main driver circuit 200 to the light-sensing scan driver 120 depending on the position of the light-sensing scan driver 120. The light-sensing control line SCL may provide the light-sensing control signal received from the main driver circuit 200 to the light-sensing scan driver 120.

The subsidiary area SBA may include the main driver circuit 200, a display pad area DPA, and first and second touch pad areas TPA1 and TPA2. The display pad area DPA, the first touch pad area TPA1, and the second touch pad area TPA2 may be disposed on the edge of the subsidiary area SBA. The display pad area DPA, the first touch pad area TPA1, and the second touch pad area TPA2 may be electrically connected to the circuit board 300 using a low-resistance, high-reliability material such as an anisotropic conductive layer and a SAP.

The fan-out lines FOL may extend from the main driver circuit 200 to the display area DA. In addition, the fan-out lines FOL may be connected so that the data voltage received from the main driver circuit 200 may be applied to the plurality of data lines DL.

The main driver circuit 200 may output signals and voltages for driving the display panel 100 to the fan-out lines FOL. The main driver circuit 200 may provide data voltages to the data lines DL through the fan-out lines FOL. The data voltages may be applied to the plurality of pixels SP, so that the luminance of the plurality of display pixels SP may be determined. The main driver circuit 200 may supply a scan control signal to the display scan driver 110 through the gate control line GCL.

The main driver circuit 200 may receive light-sensing signals from light-sensing pixels LSP through the light-sensing lines RLn, and may detect photoplethysmography signals among biological signals associated with changes in the magnitude of the light-sensing signals. The light-sensing signals may be a pulse wave signal.

The biological signals may further include electromyography signals, or brain wave signals, in addition to pulse wave signals. In the present description, the main driver circuit 200 detects and analyzes pulse wave signals among the biological signals to measure the user's biometric information as an example. The user's biometric information may include information such as blood pressure, heart rate, heart rate variability, respiratory rate, blood vessel elasticity, cardiovascular disease, or oxygen saturation.

The main driver circuit 200 may guide a process of detecting pulse wave signals using the application on the screen so that pulse wave signals can be detected from multiple fingers of the user through the first and second biometric information measurement areas FSA1 and FSA2. For example, the first and second biometric information measurement areas FSA1 and FSA2 may each detect a pulse wave signal from a respective finger of the user. The main driver circuit 200 may detect pulse wave signals in real time through left and right fingers through the first and second biometric information measurement areas FSA1 and FSA2. Accordingly, the main driver circuit 200 may compare the pulse wave signals detected through the left and right fingers, and may select one of the pulse wave signals based on characteristics thereof. For example, the main driver circuit 200 may select one of the pulse wave signals based on amplitude or pulse width. For example, a selected pulse wave signal may have a greater amplitude or pulse width among the pulse wave signals. The main driver circuit 200 may detect numerical values of the characteristic information of the selected pulse wave signal, such as an amplitude or a pulse width range (numerical range), systole and diastole periods, systolic blood pressure change width range (numerical range), systolic period, or diastolic period change range (numerical range).

The main driver circuit 200 may measure biometric information such as blood pressure, heart rate, or heart rate variability by analyzing characteristic information of pulse wave signals selected and detected in real time through a predetermined machine learning algorithm. The main driver circuit 200 may display the measured biometric information using an application program in the display area DA.

On the other hand, the main driver circuit 200 may generate digital video data according to the touch coordinates based on the touch coordinate data from the touch driver circuit 400 or may execute an application program indicated by an icon displayed at the user's touch coordinates.

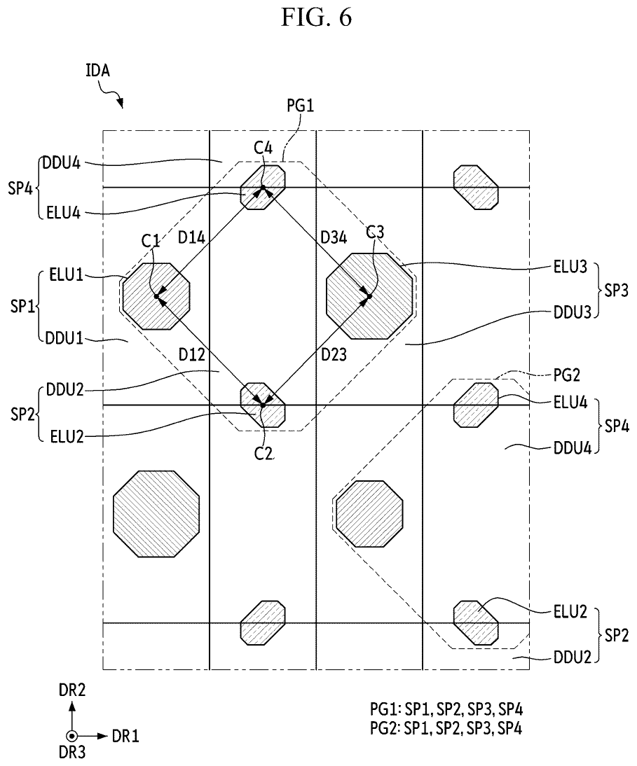

FIG. 6 is a layout view showing arrangement structure of pixels in an image display area of a display area according to an embodiment.

Referring to FIG. 6, display pixels SP configured to display images may be disposed in the image display area IDA of the display area DA. Infrared light-emitting pixels and light-sensing pixels LSP may not be formed in the image display area IDA of the display area DA. The display pixels SP may be divided into first display pixels SP1, second display pixels SP2, third display pixels SP3 and fourth display pixels SP4.

In the image display area IDA, a first unit pixel PG1 may be configured to display images. The first unit pixel PG1 may include a first display pixel SP1, a second display pixel SP2, and a third display pixel SP3, The first unit pixel PG1 may further include a fourth display pixel SP4. The first unit pixels PG1 in the image display area IDA may be defined as the minimum unit of display pixels capable of representing white light.

The first unit pixels PG1 in the image display area IDA may be arranged in a matrix pattern along the first direction DR1 and the second direction DR2. In other words, the first unit pixels PG1 in the image display area IDA may be arranged alternately in horizontal or vertical stripes to form a matrix pattern.

The first display pixel SP1 may include a first light-emitting unit ELU1 that may emit first light and a first pixel driving unit DDU1 that may apply a driving current to the light-emitting element of the first light-emitting unit ELUL. The first light may be light in a red wavelength range. For example, the main peak wavelength of the first light may be located between approximately 600 nm and 750 nm.

The second display pixel SP2 may include a second light-emitting unit ELU2 that may emit second light and a second pixel driving unit DDU2 that may apply a driving current to the light-emitting element of the second light-emitting unit ELU2. The second light may be light in a blue wavelength range. For example, a main peak wavelength of the second light may be located between approximately 370 nm and 460 nm.

The third display pixel SP3 may include a third light-emitting unit ELU3 that may emit third light and a third pixel driving unit DDU3 that may apply a driving current to the light-emitting element of the third light-emitting unit ELU3. The third light may be light in a blue wavelength range. For example, the third light may be light in a green wavelength range. For example, the main peak wavelength of the third light may be located between approximately 480 nm and 560 nm.

The fourth display pixel SP4 may include a fourth light-emitting unit ELU4 that may emit fourth light and a fourth pixel driving unit DDU4 that may apply a driving current to the light-emitting element of the fourth light-emitting unit ELU4. The fourth light may be light in a white wavelength range. Alternatively, the fourth light may be light in the same wavelength range as the second or third light.

In the first unit pixel GP1, the first to third pixel driving units DDU1 to DDU3 may be arranged in a predetermined order in the first direction DR1. Alternatively, one of the first to third pixel driving units DDU1 to DDU3 may be arranged in the first direction DR1 with another adjacent pixel driving unit. In addition, the fourth pixel driving unit DDU4 may be arranged in the first direction DR1 with one of the first to third pixel driving units DDU1 to DDU3. Alternatively, the fourth pixel driving unit DDU4 may be arranged in the second direction DR2 with one of the first to third pixel driving units DDU1 to DDU3.

The first pixel driving units DDU1 adjacent to one another in a data line direction may be arranged in the second direction DR2. The second pixel driving units DDU2 adjacent to one another in the data line direction may be arranged in the second direction DR2. Likewise, the fourth pixel driving units DDU4 adjacent to one another in the data line direction may be arranged in the second direction DR2.

The first light-emitting unit ELU1, the second light-emitting unit ELU2, the third light-emitting unit ELU3 and the fourth light-emitting unit ELU4 may have, but is not limited to, a rectangular shape, an octagonal shape or a diamond shape when viewed from the top. The first light-emitting unit ELU1, the second light-emitting unit ELU2, the third light-emitting unit ELU3, and the fourth light-emitting unit ELU4 may have other polygonal shapes than a rectangular shape, an octagonal shape or a diamond shape when viewed from the top.

Due to the arrangement positions and planar shapes of the first light-emitting unit ELU1, the second light-emitting unit ELU2, the third light-emitting unit ELU3, and the fourth light-emitting unit ELU4, the distance D12 between the center C1 of the first light-emitting unit ELU1 and the center C2 of the second light-emitting unit ELU2, the distance D23 between the center C2 of the second light-emitting unit ELU2 and the center C3 of the third light-emitting unit ELU3 which may be adjacent each other, the distance D14 between the center C1 of the first light-emitting unit ELU1 and the center C4 of the fourth light-emitting unit ELU4 which may be adjacent each other in another direction, and the distance D34 between the center C3 of the third light-emitting unit ELU3 and the center C4 of the fourth light-emitting unit ELU4 may be substantially equal.

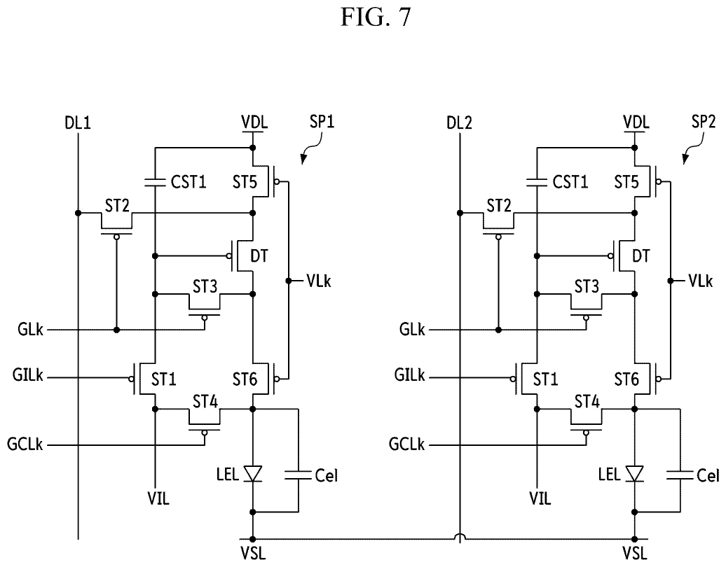

FIG. 7 is a circuit diagram showing first and second display pixels arranged in the image display area of FIG. 6.

Referring to FIG. 7, each of the display pixels SPX according to an embodiment, for example, each of the first and second display pixels SP1 and SP2 may be connected to the k^{th} display initialization line GILk, the k^{th} display scan line GLk, the k^{th} display control line GCLk, and the k^{th} emission control line VLk. The first display pixel SP1 may be connected to a first supply voltage line VDL from which a first supply voltage is supplied, a second supply voltage line VSL from which a second supply voltage is supplied, and a third supply voltage line VIL from which a third supply voltage is supplied. In the present description, the letters such as k and n used in place of numbers may be defined as positive integers excluding zero.

The first light-emitting unit ELU1 of the first display pixel SP1 may include a light-emitting element LEL. The first pixel driving unit DDU1 may include a driving transistor DT, switch elements, and a capacitor CST1. The switch elements include first to sixth transistors ST1, ST2, ST3, ST4, ST5 and ST6.

The driving transistor DT may include a gate electrode, a first electrode, and a second electrode. A drain-source current Ids (hereinafter referred to as “driving current”) of driving transistor DT flowing between the first electrode and the second electrode may be controlled according to the data voltage applied to the gate electrode. The driving current Ids flowing through the channel of the driving transistor DT may be proportional to the square of the difference between a voltage Vgs between the first electrode and the gate electrode of the driving transistor DT and the threshold voltage, as shown in Equation 1:

$$Ids = {k}^{′} \times {( Vsg - Vth )}^{-} \qquad [ Equation 1 ]$$

where k′ denotes a proportional coefficient determined by the structure and physical properties of the driving transistor, Vsg denotes the voltage between the first electrode and the gate electrode of the driving transistor, and Vth denotes the threshold voltage of the driving transistor.

The light-emitting element LEL may emit light as the driving current Ids flows therein. The amount of the light emitted from the light-emitting elements LEL may increase with the driving current Ids.

The light-emitting element LEL may be an organic light-emitting diode including an organic emissive layer disposed between an anode electrode and a cathode electrode. Alternatively, the light-emitting element LEL may be an inorganic light-emitting element including an inorganic semiconductor disposed between an anode electrode and a cathode electrode. Alternatively, the light-emitting element LEL may be quantum-dot light-emitting element including a quantum-dot emissive layer disposed between an anode electrode and a cathode electrode. Alternatively, the light-emitting element LEL may be a micro light-emitting element including a micro light-emitting diode disposed between an anode electrode and a cathode electrode.

The anode electrode of the light-emitting element LEL may be connected to the first electrode of the fourth transistor ST4 and the second electrode of the sixth transistor ST6. The cathode electrode of the light-emitting element LEL may be connected to the second supply voltage line VSL. A parasitic capacitance Cel may be formed between the anode electrode and the cathode electrode of the light-emitting element LEL.

The first transistor T1 may be turned on by an initialization scan initialization signal of the k^{th} display initialization line GILk to connect the gate electrode of the driving transistor DT with the third supply voltage line VILL. Accordingly, a third supply voltage VINT1 of the third supply voltage line VIL1 may be applied to the gate electrode of the driving transistor DT. The gate electrode of the first transistor ST1 may be connected to the k^{th} display initialization line GILk, the first electrode thereof may be connected to the gate electrode of the driving transistor DT, and the second electrode thereof may be connected to the third driving voltage line VIL.

The second transistor ST2 may be turned on by the display scan signal of the k^{th} display scan line GLk to connect the first electrode of the driving transistor DT with the first data line DL1. Accordingly, the data voltage of the first data line DL1 may be applied to the first electrode of the driving transistor DT. The gate electrode of the second transistor ST2 may be connected to the k^{th} display scan line GLk, a first electrode thereof may be connected to the first electrode of the driving transistor DT, and a second electrode thereof may be connected to the first data line DL1.

Alternatively, the second transistor ST2 formed in the second display pixel SP2 may be turned on by the display scan signal of the k^{th} display scan line GLk to connect the first electrode of the driving transistor DT with the second data line DL2. Accordingly, the data voltage of the second data line DL2 may be applied to the first electrode of the driving transistor DT formed in the second display pixel SP2. The gate electrode of the second transistor ST2 may be connected to the k^{th} display scan line GLk, a first electrode thereof may be connected to the first electrode of the driving transistor DT, and a second electrode thereof may be connected to the second data line DL2.

The third transistor ST3 may be turned on by the display scan signal of the k^{th} display scan line GLk to connect the gate electrode with the second electrode of the driving transistor DT. When the gate electrode and the second electrode of the driving transistor DT are connected with each other, the driving transistor DT may function as a diode. A gate electrode of the third transistor ST3 may be connected to the k^{th} display scan line GLk, a first electrode thereof may be connected to the second electrode of the driving transistor DT, and a second electrode thereof may be connected to the gate electrode of the driving transistor DT.

The fourth transistor ST4 may be turned on by a display control signal of the kl display control line GCLk to connect the anode electrode of the light-emitting element LEL with the third supply voltage line VIL. The third supply voltage of the third supply voltage line VIL may be applied to the anode electrode of the light-emitting element LEL. The gate electrode of the fourth transistor ST4 may be connected to the k^{th} display control line GCLk, the first electrode thereof may be connected to the anode electrode of the light-emitting element LEL, and the second electrode thereof may be connected to the third supply voltage line VIL.

The fifth transistor ST5 may be turned on by the emission signal of a k^{th} emission control line VLk to connect the first electrode of the driving transistor DT with the first supply voltage line VDL. The gate electrode of the fifth transistor ST5 is connected to the k^{th} emission control line VLk, the first electrode thereof is connected to the first supply voltage line VDL, and the second electrode thereof is connected to the first electrode of the driving transistor DT.

The sixth transistor ST6 is disposed between the second electrode of the driving transistor DT and the anode electrode of the light-emitting element LEL. The sixth transistor ST6 may be turned on by the emission control signal of the k^{th} emission control line VLk to connect the second electrode of the driving transistor DT with the anode electrode of the light-emitting element LEL. The gate electrode of the sixth transistor ST6 is connected to the ki emission control line VLk, the first electrode thereof is connected to the second electrode of the driving transistor DT, and the second electrode thereof is connected to the anode electrode of the light-emitting element LEL.

When both the fifth transistor ST5 and the sixth transistor ST6 are turned on, the driving current Ids of the driving transistor DT according to the data voltage applied the gate electrode of the driving transistor DT may flow to the light-emitting element LEL.

The capacitor CST1 may be formed between the gate electrode of the driving transistor DT and the first supply voltage line VDL. The first capacitor electrode of the capacitor CST1 may be connected to the gate electrode of the driving transistor DT, and the second capacitor electrode thereof may be connected to the first driving voltage line VDL.

When the first electrode of each of the first to sixth transistors ST1, ST2, ST3, ST4, ST5 and ST6 and the driving transistor DT is a source electrode, the second electrode thereof may be a drain electrode. Alternatively, when the first electrode of each of the first to sixth transistors ST1, ST2, ST3, ST4, ST5 and ST6 and the driving transistor DT is a drain electrode, the second electrode thereof may be a source electrode.

The active layer of each of the first to sixth transistors ST1, ST2, ST3, ST4, ST5 and ST6 and the driving transistor DT may be made of at least one of poly silicon, amorphous silicon, or oxide semiconductor. Although the first to sixth transistors ST1, ST2, ST3, ST4, ST5 and ST6 and the driving transistor DT are implemented as p-type metal oxide semiconductor field effect transistors (MOSFETs) in FIG. 7, this is merely illustrative. For example, the first to sixth transistors ST1, ST2, ST3, ST4, ST5 and ST6, and the driving transistor DT may be implemented as n-type MOSFETs. Alternatively, at least one of the first to sixth transistors ST1, ST2, ST3, ST4, ST5 or ST6 may be implemented as an n-type MOSFET.

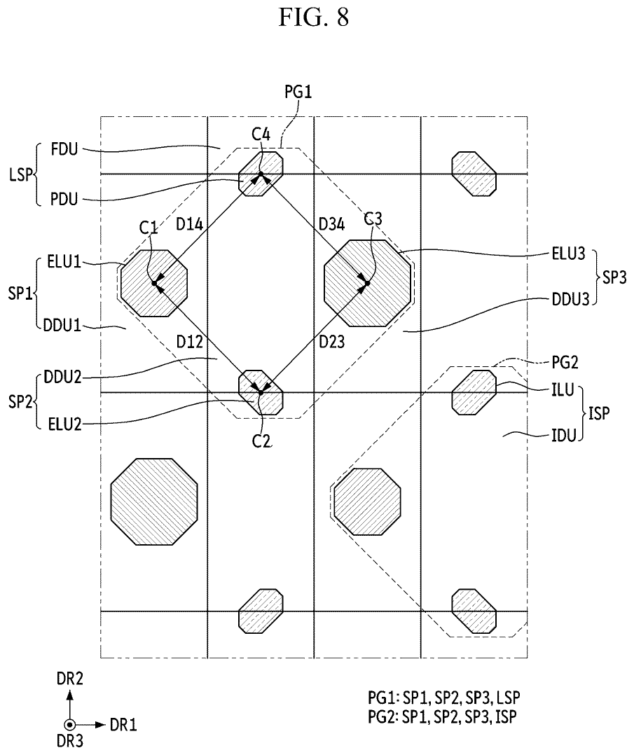

FIG. 8 is a layout view showing arrangement structure of pixels in a biometric information measurement area of a display area according to an embodiment.

Referring to FIG. 8, each of the biometric information measurement areas FSA1 and FSA2 of the display area DA may include display pixels SP, infrared light-emitting pixels ISP, and light-sensing pixels LSP. The display pixels SP may be divided into first display pixels SP1, second display pixels SP2 and third display pixels SP3.

In each of the biometric information measurement areas FSA1 and FSA2, a light-sensing pixel LSP along with a first display pixel SP1, a second display pixel SP2 and a third display pixel SP3 may be defined as a first unit pixel PG1. In addition, an infrared light-emitting pixel ISP along with a first display pixel SP1, a second display pixel SP2 and a third display pixel SP3 may be defined as a second unit pixel PG2.

Each of the first and second unit pixels PG1 and PG2 may be defined as the minimum unit of display pixels capable of representing white light. Each first unit pixel PG1 is able to sense light. The first unit pixels PG1 and the second unit pixels PG2 may be alternately arranged in a zigzag pattern when viewed from the top. The first unit pixels PG1 and the second unit pixels PG2 may be arranged in a diagonal direction in a matrix pattern. In addition, the first unit pixels PG1 and the second unit pixels PG2 may be arranged alternately in horizontal or vertical stripes to form a matrix pattern when viewed from the top.

An infrared light-emitting pixel ISP may include an infrared light-emitting unit ILU that may emit light in the infrared wavelength range, and an infrared pixel driving unit IDU for applying driving current to the light-emitting element of the infrared light-emitting unit ILU. The main peak wavelength of infrared light may lie between approximately 750 nm to 1 mm.

The light-sensing pixel LSP may include a photo-detecting unit PDU and a sense driving unit FDU.

In the first unit pixel GP1, the first to third pixel driving units DDU1 to DDU3 may be arranged in a predetermined order in the first direction DR1. Alternatively, a pixel driving unit of the first to third pixel driving units DDU1 to DDU3 may be arranged in the first direction DR1 with another adjacent pixel driving unit. In addition, the sense driving unit FDU may be arranged in the first direction DR1 of a pixel driving unit of the first to third pixel driving units DDU1 to DDU3. Alternatively, the sense driving unit FDU may be arranged in the second direction DR2 of a pixel driving unit of the first to third pixel driving units DDU1 to DDU3.

The first pixel driving units DDU1 adjacent to one another in a data line direction may be arranged in the second direction DR2. The second pixel driving units DDU2 adjacent to one another in the data line direction may be arranged in the second direction DR2. Likewise, all of the sense driving units FDU adjacent to one another in the data line direction may be arranged in the second direction DR2.

The first light-emitting unit ELU1, the second light-emitting unit ELU2, the third light-emitting unit ELU3, the infrared light-emitting unit ILU and the photo-detecting unit PDU may have, but is not limited to, a rectangular shape, an octagonal shape or a diamond shape when viewed from the top. The first light-emitting unit ELU1, the second light-emitting unit ELU2, the third light-emitting unit ELU3, the infrared light-emitting unit ILU and the photo-detecting unit PDU may have other polygonal shapes than a rectangular shape, an octagonal shape or a diamond shape when viewed from the top.

Due to the arrangement positions and planar shapes of the first light-emitting unit ELU1, the second light-emitting unit ELU2, the third light-emitting unit ELU3 and the fourth light-emitting unit ELU4, the distance D12 between the center C1 of the first light-emitting unit ELU1 and the center C2 of the second light-emitting unit ELU2, the distance D23 between the center C2 of the second light-emitting unit ELU2 and the center C3 of the third light-emitting unit ELU3 which may be adjacent each other, the distance D14 between the center C1 of the first light-emitting unit ELU1 and the center C4 of the photo-detecting unit PDU which may be adjacent each other in another direction, and the distance D34 between the center C4 of the photo-detecting unit PDU and the center C3 of the third light-emitting unit ELU3 may be substantially equal.

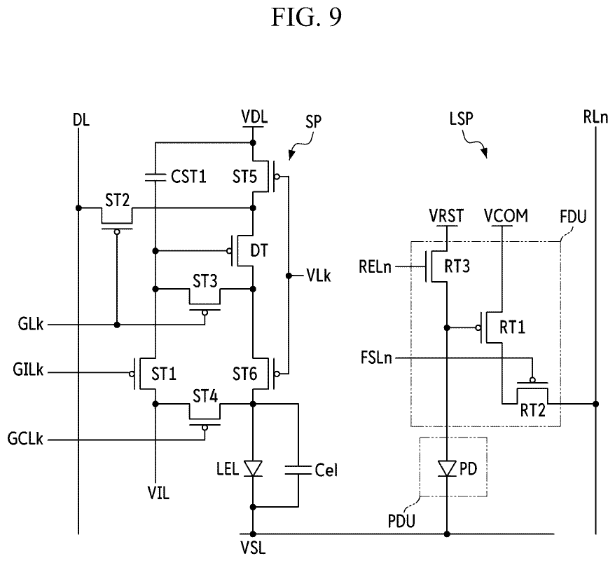

FIG. 9 is a circuit diagram showing a display pixel and a light-sensing pixel disposed in the biometric information measurement area of FIG. 8.

The circuit structure of the display pixels SP formed in the biometric information measurement areas FSA1 and FSA2 may be substantially identical to the circuit structure of the first and second display pixels SP1 and SP2 described above with reference to FIG. 6; and redundant descriptions thereof may be omitted or simplified.

It should be noted that the light-sensing pixels LSP formed in the biometric information measurement areas FSA1 and FSA2 may be electrically connected to the n^{th} sense reset line RSLn, the n^{th} light-sensing scan line FSLn and the n^{th} light-sensing line RLn. Each of the light-sensing pixels LSP may be reset by a reset signal from the n^{th} sensing reset line RSLn, and may transmit a light-sensing signal to the n^{th} light-sensing line RLn in response to the sensing scan signal from the n^{th} light-sensing scan line FSLn.

The light-sensing pixels LSP may be divided into a photo-detecting unit PDU including a photo-detecting element PD, and a sense driving unit FDU including first to third sensing transistors RT1 to RT3 and a sensing capacitor (not shown). The sensing capacitor may be formed in parallel with the photo-detecting element PD.

A first sensing transistor RT1 of the sense driving unit FDU may allow a light-sensing current to flow according to the voltages of the photo-detecting element PD and the sensing capacitor. The amount of current of the light-sensing current may vary depending on a voltage applied to the photo-detecting element PD and the sensing capacitor. The gate electrode of the first sensing transistor RT1 may be connected to the second electrode of the photo-detecting element PD. A first electrode of the first sensing transistor RT1 may be connected to a common voltage source VCOM from which a common voltage may be applied. A second electrode of the first sensing transistor RT1 may be connected to a first electrode of the second sensing transistor RT2.

When the sensing scan signal of the gate-on voltage is applied to the n^{th} light-sensing scan line FSL, the second sensing transistor RT2 allows the sensing current of the first sensing transistor RT1 to flow to the n^{th} light-sensing line RLn. In this example, the n^{th} light-sensing line RLn may be charged with the sensing voltage by the sensing current. The gate electrode of the second sensing transistor RT2 may be connected to the n^{th} light-sensing scan line FSL, the first electrode thereof may be connected to the second electrode of the first sensing transistor RT1, and the second electrode thereof may be connected to the n^{th} light-sensing line RLn.

When a reset signal of the gate-on voltage is applied to the n^{th} sensing reset line RSLn, the third sensing transistor RT3 may reset the voltages of the photo-detecting element PD and the sensing capacitor to the reset voltage of a reset voltage source VRST. The gate electrode of the third sensing transistor RT3 may be connected to the sensing reset line RSL, the first electrode thereof may be connected to the reset voltage source VRST, and the second electrode thereof may be connected to the second electrode of the photo-detecting element PD.

Although the first sensing transistor RT1 and the second sensing transistor RT2 may be implemented as p-type metal oxide semiconductor field effect transistors (MOSFETs) while the third sensing transistor RT3 may be implemented as an n-type MOSFET in the example shown in FIG. 9, this is merely illustrative. Optionally, the first sensing transistor RT1 and the second sensing transistor RT2 may be of the same type or different types. In addition, one of the first or second electrodes of each of the first sensing transistor RT1, the second sensing transistor RT2 and the third sensing transistor RT3 may be a source electrode, while the other one may be a drain electrode.

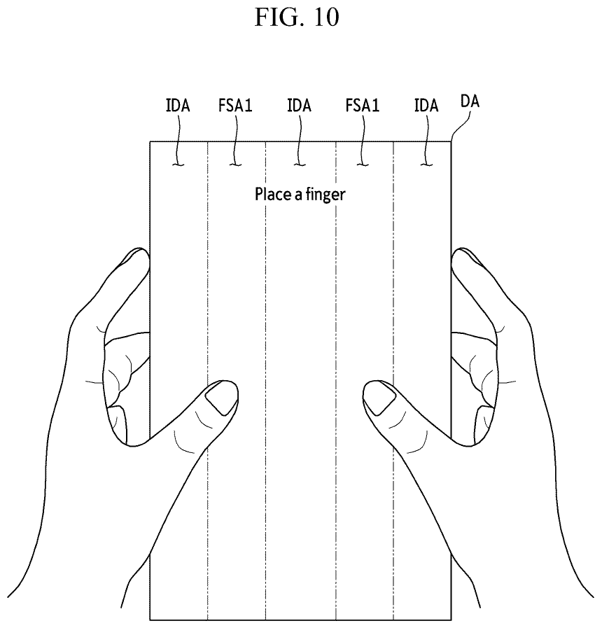

FIG. 10 is a view showing a method of measuring pulse wave signals at a plurality of touch locations through a plurality of biometric information measurement areas.

The first and second biometric information measurement areas FSA1 and FSA2 may have a constant width in the width direction of the display panel 100 (e.g., the first direction DR1) and may extend in the length direction of the display panel 100 (e.g., the second direction DR2). The width of the first and second biometric information measurement areas FSA1 and FSA2 may be less than a length thereof in length direction of the display panel 100. The first and second biometric information measurement areas FSA1 and FSA2 may be formed parallel to each other in the length direction of the display panel 100 where the main driver circuit 200 is disposed. The first and second biometric information measurement areas FSA1 and FSA2 may be equally spaced apart from each other between adjacent image display areas IDA.

During the biometric information measurement period, the main driver circuit 200 may display an application program and the first and second biometric information measurement areas FSA1 and FSA2 in the display area DA, which may guide a process of detecting a user's finger touch and pulse waves.

Specifically, the main driver circuit 200 may control the scan signal output from the display scan driver 110 and the light-sensing scan driver 120, and may provide a data signal of a preset magnitude to the display pixels SP or infrared light-emitting pixels ISP formed in the first and second biometric information measurement areas FSA1 and FSA2, and the display pixels SP and the infrared light-emitting pixels ISP formed in the first and second biometric information measurement areas FSA1 and FSA2 may be driven. The main driver circuit 200 may drive the light-sensing pixels LSP formed in the first and second biometric information measurement areas FSA1 and FSA2 to receive light-sensing signals through the light-sensing pixels LSP.

The main driver circuit 200 may use light-sensing signals received through light-sensing pixels LSP arranged in the first and second biometric information measurement areas FSA1 and FSA2 to determine pulse wave signals reflecting blood changes according to heartbeat, and measure the user's biometric information such as blood pressure according to the magnitude and change cycle of the pulse wave signals. Subsequently, the main driver circuit 200 may generate result image data based on biometric information measurement results measured in real time and display the result image data on the display panel 100 as an image.

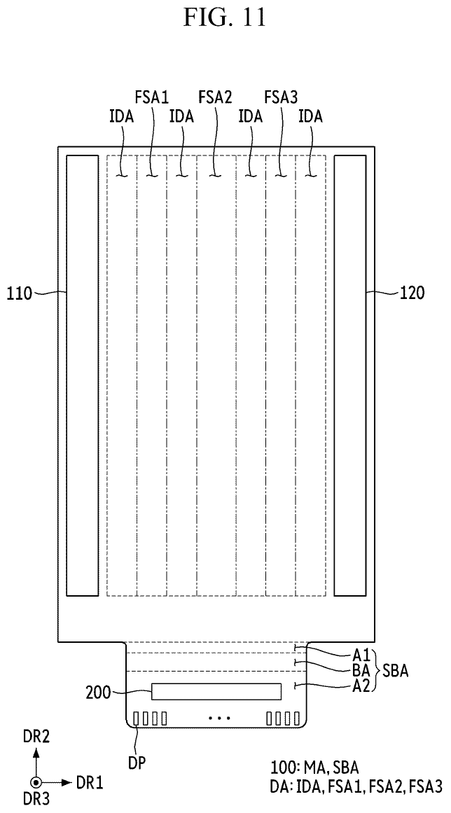

FIG. 11 is a plan view showing an arrangement structure of a display panel and a main driver circuit shown in FIG. 1 according to an embodiment.

Referring to FIG. 11, in the display area DA, biometric information measurement areas FSA1, FSA2 and FSA3 including display pixels SP and light-sensing pixels LSP may be formed and divided into at least three areas.

Specifically, the first to third biometric information measurement areas FSA1, FSA2 and FSA3 may each be formed in a rectangular shape having longer sides in the longitudinal direction of the display panel 100 (e.g., in the second direction DR2). In particular, the first to third biometric information measurement areas FSA1, FSA2 and FSA3 may be formed parallel to one another in the length direction of the display panel 100 where the main driver circuit 200 is disposed, and may be equally spaced apart from one another between adjacent image display areas IDA.

The main driver circuit 200 may be disposed at a side of the display area DA, which may be the side of the display area DA in the length direction, and may be disposed on the circuit board 300 electrically connected to the side of the display area DA.

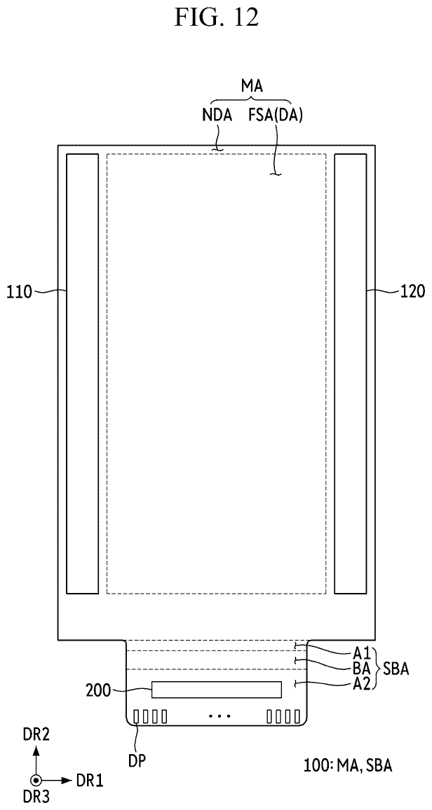

FIG. 12 is a plan view showing an arrangement structure of a display panel and a main driver circuit shown in FIG. 1 according to an embodiment.

Referring to FIG. 12, in the display area DA of the display panel 100, display pixels SP configured to display images, light-sensing pixels LSP configured to sense light, and infrared light-emitting pixels ISP may all be included. In other words, the display pixels SP, the light-sensing pixels LSP, and the infrared light-emitting pixels ISP may be arranged sequentially and repeatedly in a predetermined order throughout the display area DA.

It should be noted that the light-sensing lines RLn of the light-sensing pixels LSP disposed in one or more predetermined biometric information measurement areas FSA1, FSA2 and FSA3 in the display area DA may be electrically connected to the main driver circuit 200.

The n^{th} light-sensing lines RLn of the light-sensing pixels LSP disposed in the image display areas IDA, other than the one or more biometric information measurement areas FSA1, FSA2 and FSA3, may be disconnected from the main driver circuit 200.

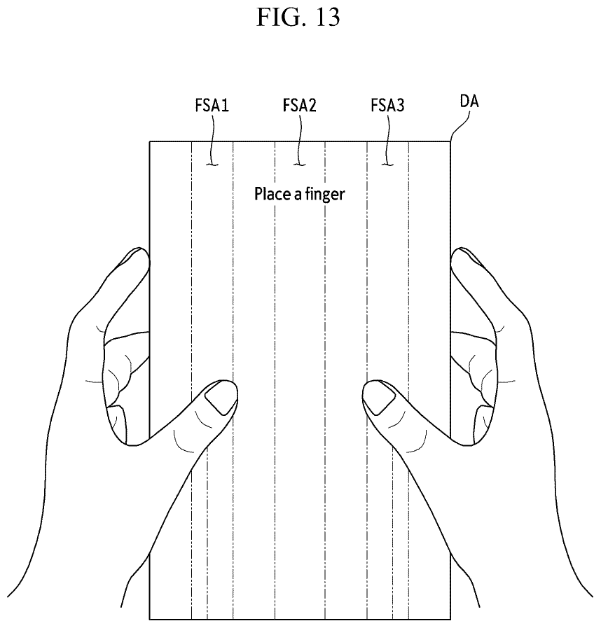

FIG. 13 is a view showing a method of measuring pulse wave signals at a plurality of touch locations through a plurality of biometric information measurement areas.

Referring to FIG. 13, the first to third biometric information measurement areas FSA1, FSA2 and FSA3 may have a constant width in the width direction of the display panel 100 (e.g., the first direction DR1) and may extend in the length direction of the display panel 100 (e.g., the second direction DR2). The width may be less than a length of the first to third biometric information measurement areas FSA1, FSA2 and FSA3 in the length direction of the display panel 100. The first to third biometric information measurement areas FSA1, FSA2 and FSA3 may be formed parallel to each other in the length direction of the display panel 100 where the main driver circuit 200 is disposed, and may be equally spaced apart from each other between adjacent image display areas IDA.

During the biometric information measurement period, the main driver circuit 200 may displays an application program and the first to third biometric information measurement areas FSA1, FSA2 and FSA3 in the display area DA, which may guide a process of detecting a user's finger touch and pulse waves. In doing so, the main driver circuit 200 may display the period for measuring biometric information, the period for detecting pulse wave signals, and the first to third biometric information measurement areas FSA1, FSA2 and FSA3 in the form of bars using the application program on the screen. It is, however, to be understood that the present disclosure is not limited thereto. For example, other graphics or text may be used to convey information.

Subsequently, the main driver circuit 200 may apply data voltage to the first and second unit pixels PG1 and PG2 arranged in a plurality of biometric information measurement areas FSA1, FSA2 and FSA3, and may supply control signals to the display scan driver 110 and the light-sensing scan driver 120. At this time, the main driver circuit 200 may apply a predetermined data voltage to at least one display pixel among the first and second display pixels SP1 and SP2 included in the first and second unit pixels PG1 and PG2, so that an optical signal can be detected by at least one of green light or red light. Alternatively, an optical signal by infrared light may be detected by applying a predetermined data voltage to the infrared light-emitting pixels ISP. Subsequently, the main driver circuit 200 may receive optical signals from the light-sensing pixels LSP arranged in each of the biometric information measurement areas FSA1 and FSA2 through the light-sensing lines RLn of the display panel 100, i.e., light-sensing signals.

The main driver circuit 200 may detect first and second pulse wave signals associated with the light-sensing signals for each of the biometric information measurement areas FSA1, FSA2 and FSA3 received in real time, and may store them as digital signal data. The pulse wave signals may be associated with the magnitude and changes in the magnitude of the light-sensing signals.

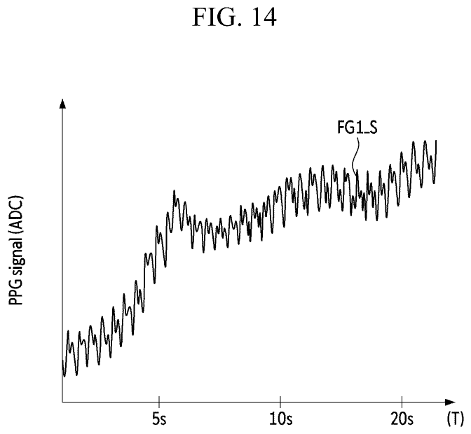

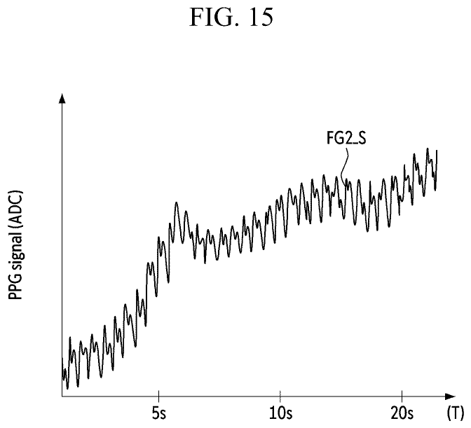

FIG. 14 is a waveform diagram showing a pulse wave signal of a finger detected in the first biometric information measurement area. FIG. 15 is a waveform diagram showing a pulse wave signal of a finger detected in the second biometric information measurement area.

Referring to FIG. 14 and FIG. 15, the main driver circuit 200 receives first and second pulse wave signals PG1_S and PG2_S in real time through the light-sensing pixels LSP and light-sensing lines RLn for each of the biometric information measurement areas FSA1 to FSA3.

Characteristics such as pulse width and amplitude of the first and second pulse wave signals PG1_S and PG2_S may be detected differently.

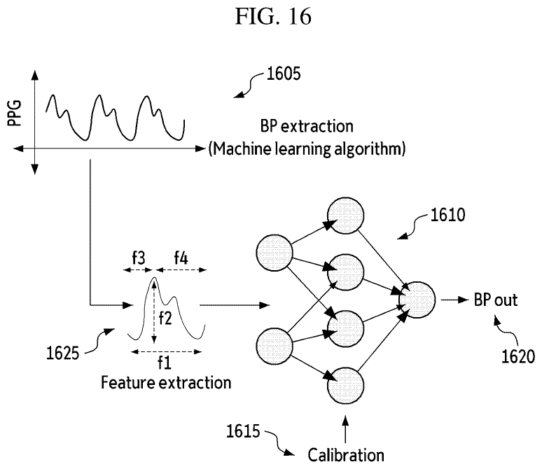

FIG. 16 is a diagram for illustrating a method of calculating blood pressure information using a machine learning algorithm according to an embodiment.

Referring to FIG. 16, the main driver circuit 200 may compare the first and second pulse wave signals PG1_S and PG2_S and select one of the pulse wave signals PG1_S and PG2_S based on the amplitude or pulse width. Then, the selected pulse wave signal of the pulse wave signals PG1_S and PG2_S may be analyzed through a predetermined machine learning algorithm.

For example, the main driver circuit 200 may compare the characteristic change information of a pulse wave signal (e.g., the first pulse wave signal PG1_S) with the reference pulse wave characteristic information set by predetermined experimental values, and may generate comparison results. Then, the main driver circuit 200 may build a database including the comparison results as learning data. The main driver circuit 200 may compare changes in characteristics according to the first pulse wave signals PG1_S, i.e., a pulse width f1 (e.g., systole and diastole periods) and number information, a systolic amplitude f2 (e.g., systolic blood pressure value), information on changes in systolic pulse width f3 (e.g., systolic period) and information on changes in diastolic pulse width f4 (e.g., diastolic period) with reference pulse wave characteristic information, i.e., a pulse width f1 (e.g., systole and diastole periods) and number information, a systolic amplitude f2 (e.g., systolic blood pressure value), information on changes in systolic pulse width f3 (e.g. systolic period) and information on changes in diastolic pulse width f4 (e.g., diastolic period). Then, the main driver circuit 200 may build a database including the comparison results as learning data 1605. The main driver circuit 200 may use a machine learning algorithm to train a model 1610 using the learning data 1605. For example, the model 1610 may be a neural network model, which may include a plurality of layers of interconnected nodes. Each node may be a computational unit with at least one weighted input, a transfer function that combines inputs, and an output. The nodes may be organized into connected layers. These layers may extract features, transform data, and make predictions. Optionally, the main driver circuit 200 may use calibration input 1615 to update the model 1610. For example, the calibration input 1615 may be used to adjust weights on the nodes.

The main driver circuit 200 may use the model 1610 to calculate first biometric information 1620 on features extracted from measured biometric data 1625. For example, the main driver circuit 200 may use the model 1610 to calculate first biometric information 1620 on at least one of blood pressure, heart rate, heart rate variability, respiratory rate, vascular elasticity, cardiovascular disease analysis results, or oxygen saturation, based on the change rate or change amount of the characteristic information of the first pulse wave signals PG1_S compared to the reference pulse wave characteristic information. In addition, the main driver circuit 200 may use the model 1610 to analyze a high pulse period and changes in the high pulse period of the first pulse wave signals PG1_S, a magnitude and changes in the magnitude of the high pulse, a magnitude and changes in the magnitude of a low pulse, high pulse waveform changes, a time taken to reach the peak of the high pulse, difference between the magnitudes of the pulses of first pulse wave signals FG1_S detected by green light and red light, compared to the reference pulse wave characteristic information. Then, the main driver circuit 200 may derive the first biometric information 1620 on features extracted from measured biometric data 1625, which may include at least one of blood pressure, heart rate, heart rate variability, respiratory rate, vascular elasticity, cardiovascular disease analysis results, or oxygen saturation from the analysis results.

The main driver circuit 200 may estimate the blood pressures of the finger blood vessels based on the time differences between the time points corresponding to the peak of the first pulse wave signals FG1_S and the time points corresponding to peaks of filtered pulse waves. For example, before and after the time points corresponding to the peaks of the first pulse wave signals FG1_S, the main driver circuit 200 may calculate pulse wave signals for a predetermined period and may detect the blood pressure based on differences in the pulse wave signals. Among the estimated blood pressures, the highest blood pressure may be calculated as the systolic blood pressure while the lowest blood pressure may be calculated as the diastolic blood pressure. In addition, other blood pressures such as the average blood pressure may be calculated using the estimated blood pressures.

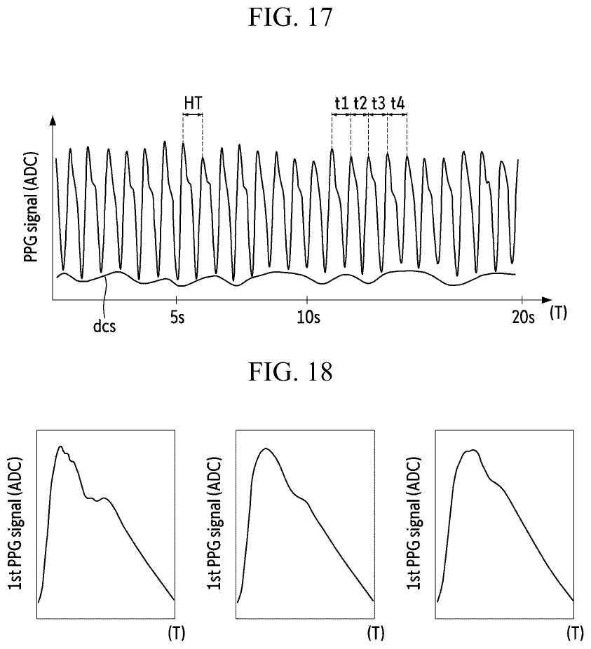

FIG. 17 is a graph for illustrating a method of calculating information about heart rate and respiration among biometric information according to an embodiment.

Referring to FIG. 17, the main driver circuit 200 may sample the first pulse wave signals FG1_S during a predetermined sampling period before and after the time points corresponding to the peaks of the first pulse wave signals FG1_S, and may detect the generation period HT of high pulses for the sampled first pulse wave signals FG1_S. In addition, the main driver circuit 200 may count the number of high pulses for each predetermined reference period (e.g., 60 seconds) for the sampled first pulse wave signals FG1_S to detect biometric information about the heart period and heart rate (HR).

In addition, the main driver circuit 200 may detect the heart period HT and heart period changes t1 to t4 of the high pulses for each predetermined reference period for the peaks of the pulse wave signals, to detect heart rate variability (HRV) according to the rate of change of the heart period.

Meanwhile, the main driver circuit 200 may sequentially detect the generation period of low pulses and the magnitudes of the low pulses for the sampled first pulse wave signals FG1_S. In addition, the main driver circuit 200 may detect the change period of the magnitudes dcs of the low pulses every predetermined reference period (e.g., 60 seconds) to detect changes in the respiration and respiratory rate (RR) of the user. In doing so, the main driver circuit 200 may analyze a period in which the magnitude dcs of low pulses rises and a period in which the magnitude dcs of low pulses falls, to detect the changes in the respiration and the respiratory rate (RR) of the user based on the periods in which the magnitude dcs of low pulses rises and falls.

FIG. 18 is a graph for illustrating a method for calculating information about blood vessel elasticity among biometric information according to an embodiment.

Referring to FIG. 18, the main driver circuit 200 may magnify and analyze fluctuations of the high pulse of the sampled first pulse wave signals FG1_S to set and derive blood vessel elasticity (BVE).

If the blood flow increases due to heartbeat, the pulse wave signal may be changed into high pulses, and if the blood flow decreases, the pulse wave signal may be changed again into low pulses. If blood flow changes rapidly due to the shape of the blood vessel while the blood flow increases or decreases, the blood flow may settle down quickly or change slowly depending on the elasticity of the blood vessel. Accordingly, the main driver circuit 200 may magnify and analyze the fluctuations of the high pulse of the first pulse wave signals FG1_S to set and derive the blood vessel elasticity (BVE) as values corresponding to the magnitude of the fluctuations of the high pulses.

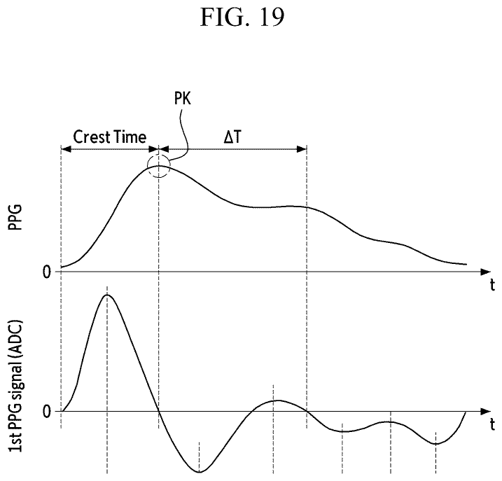

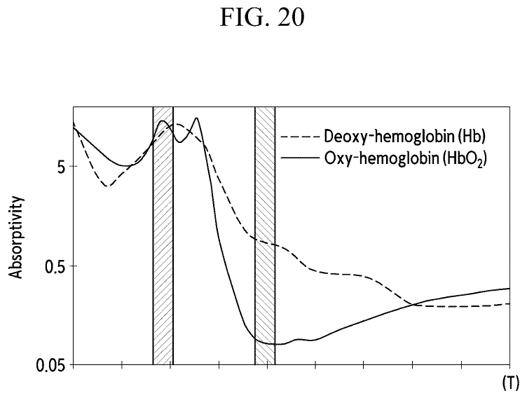

FIG. 19 is a graph for illustrating a method for calculating information about cardiovascular disease among biometric information according to an embodiment.

Referring to FIG. 19, the main driver circuit 200 may calculate a differential of, magnify and analyze the fluctuations of the high pulse of the sampled first pulse wave signals FG1_S to set and derive a score for cardiovascular disease (or a cardiovascular health analysis result score). For example, the main driver circuit 200 may detect a period Crest Time during which the first pulse wave signals FG1_S reach the peak PK in the form of high pulses, and the amount of change in time ΔT during which the pulse wave signals PPG fall relative to the period to reach the peak PK (Crest Time). The longer the period (Crest Time) during which the first pulse wave signals FG1_S reach the peak PK in the form of high pulses, the greater the risk of heart disease. Accordingly, the main driver circuit 200 may set and derive an evaluation score for cardiovascular disease (or a cardiovascular health analysis result score) inversely proportional to the period Crest Time during which the first pulse wave signals FG1_S reach the peak PK in the form of high pulses.

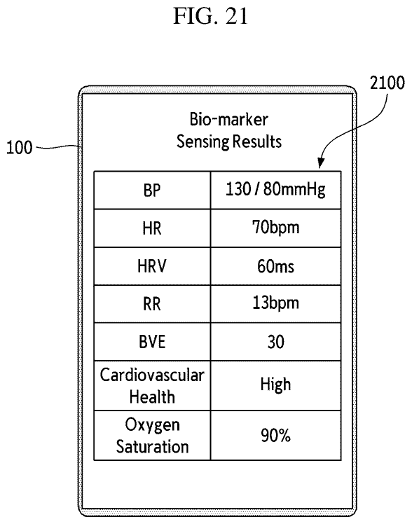

FIG. 20 is a graph for illustrating a method for calculating information about oxygen saturation among biometric information according to an embodiment.

Referring to FIG. 20, during a systole of a heart, red blood cells transport more oxyhemoglobin to peripheral tissues. On the other hand, during a diastole of the heart, the heart muscle relaxes and the chambers of the heart fill with blood, which may draw blood from peripheral tissues towards the heart. By utilizing this, the main driver circuit 200 may detect the Hb (deoxy-hemoglobin) level based on changes in the magnitude of the first pulse wave signals FG1_S detected by green light, and detects the HbO2 (oxy-hemoglobin) level based on changes in the magnitude of the first pulse wave signals FG1_S detected by red light.

The main driver circuit 200 may detect oxygen saturation (SpO₂) using Equation 2:

$${SpO}_{2} = {HpO}_{2} / {SpO}_{2} + Hb \qquad [ Equation 2 ]$$

FIG. 21 is a diagram showing the results of biometric information measurement using the display device according to an embodiment of the present disclosure.

Referring to FIG. 21, the main driver circuit 200 may generate result image data 2100 based on biometric information measurement results measured in real time and display the result image data 2100 on the display panel 100 as an image.

Specifically, the main driver circuit 200 may display biometric information such as blood pressure BP, heart rate (HR), heart rate variability (HRV), respiratory rate (RR), vascular elasticity BVE, cardiovascular disease (or cardiovascular health analysis score) and oxygen saturation (SpO₂) using an application program on the screen.

In concluding the detailed description, those skilled in the art will appreciate that many variations and modifications can be made to embodiments without substantially departing from the principles of the present disclosure. Therefore, embodiments of the disclosure are used in a generic and descriptive sense only and not for purposes of limitation.

What is claimed is:

1. A display device comprising:
a display panel having a display area divided into a plurality of image display areas and a plurality of biometric information measurement areas;
a plurality of display pixels arranged in the plurality of image display areas and the plurality of biometric information measurement areas;
a plurality of light-sensing pixels arranged in the plurality of biometric information measurement areas;
a plurality of infrared light-emitting pixels arranged in the plurality of biometric information measurement areas;
a display scan driver configured to drive the plurality of display pixels and the plurality of infrared light-emitting pixels to emit light;
a light-sensing scan driver configured to drive the plurality of light-sensing pixels to sense light; and
a main driver circuit configured to detect a pulse wave signal and measure biometric information using light-sensing signals received from the plurality of light-sensing pixels,
wherein the plurality of biometric information measurement areas are disposed in parallel rectangular shapes.

2. The display device of claim 1, wherein the main driver circuit is disposed at a side of the display area in a length direction of the display area, and receives the light-sensing signals from the plurality of light-sensing pixels at the side of the display area.

3. The display device of claim 2, wherein each of the plurality of biometric information measurement areas is extended in a length direction of the display panel toward the main driver circuit, and has a constant width in a width direction of the display panel.

4. The display device of claim 3, wherein the plurality of biometric information measurement areas are equally spaced apart from each other between the plurality of image display areas adjacent to each other.

5. The display device of claim 4, wherein a first biometric information measurement area among the plurality of biometric information measurement areas is extended in the length direction of the display area between a first image display area and a central image display area, which are extended in the length direction of the display panel, and
wherein a second biometric information measurement area among the plurality of biometric information measurement areas is extended in the length direction of the display area between the central image display area and a second image display area, which are extended in the length direction of the display panel,
wherein the first image display area and the second image display area are disposed on opposite sides of the central image display area.

6. The display device of claim 5, wherein the first image display area, the first biometric information measurement area, the central image display area, the second biometric information measurement area, and the second image display area are formed in the display area such that they are parallel to one another in the width direction and extended in the length direction of the display area.

7. The display device of claim 3, wherein the plurality of image display areas and the plurality of biometric information measurement areas are arranged in the display area of the display panel such that they are arranged alternately in a first direction, and are arranged in parallel in a second direction perpendicular to the first direction in the display area of the display panel.

8. The display device of claim 7, wherein the plurality of biometric information measurement areas each include a plurality of first unit pixels each comprising a plurality of display pixels and a light-sensing pixel, and
wherein the plurality of first unit pixels are arranged in the plurality of biometric information measurement areas in a matrix pattern in a first direction and a second direction intersecting the first direction.

9. The display device of claim 7, wherein the biometric information measurement areas each comprise:
a plurality of first unit pixels each comprising a plurality of display pixels and a light-sensing pixel; and
a plurality of second unit pixels each comprising a plurality of display pixels and an infrared light-emitting pixel,
wherein the plurality of first unit pixels and the plurality of second unit pixels are arranged alternately in the first direction and the second direction such that they are arranged in a matrix pattern in the biometric information measurement areas.

10. A display device comprising:
a display panel having a display area divided into a plurality of image display areas and a plurality of biometric information measurement areas;
a plurality of display pixels arranged in the plurality of image display areas and the plurality of biometric information measurement areas;
a plurality of light-sensing pixels arranged in the plurality of biometric information measurement areas;
a plurality of infrared light-emitting pixels arranged in the plurality of biometric information measurement areas;
a display scan driver configured to drive the plurality of display pixels and the plurality of infrared light-emitting pixels to emit light;
a light-sensing scan driver configured to drive the plurality of light-sensing pixels to sense light; and
a main driver circuit configured to detect a user's pulse wave signals and measure biometric information using light-sensing signals received from the plurality of light-sensing pixels,
wherein the plurality of image display areas and the plurality of biometric information measurement areas are arranged in the display area alternately in a first direction, and are arranged in parallel in the display area in a second direction intersecting the first direction.

11. The display device of claim 10, wherein the main driver circuit is disposed at a side of the display area in a length direction of the display area, and receives the light-sensing signals from the plurality of light-sensing pixels at the side of the display area.

12. The display device of claim 11, wherein each of the plurality of biometric information measurement areas is extended in a length direction of the display panel toward the main driver circuit, and is formed in a rectangular shape that has a constant width in a width direction of the display panel.

13. The display device of claim 12, wherein the plurality of biometric information measurement areas are disposed in parallel in the length direction of the display panel toward the main driver circuit, and are equally spaced apart from each other between the plurality of image display areas.

14. The display device of claim 13, wherein a first biometric information measurement area among the plurality of biometric information measurement areas is extended in the length direction of the display area between a first image display area and a central image display area, which are extended in the length direction of the display panel, and
wherein a second biometric information measurement area among the plurality of biometric information measurement areas is extended in the length direction of the display area between the central image display area and a second image display area, which are extended in the length direction of the display panel,
wherein the first image display area and the second image display area are disposed on opposite sides of the central image display area.

15. The display device of claim 11, wherein the plurality of biometric information measurement areas each include a plurality of first unit pixels each comprising a plurality of display pixels and a light-sensing pixel, and
wherein the plurality of first unit pixels are arranged in the plurality of biometric information measurement areas in a matrix pattern in a first direction and a second direction intersecting the first direction.

16. The display device of claim 15, wherein the plurality of biometric information measurement areas each include a plurality of first unit pixels each comprising a plurality of display pixels and a light-sensing pixel, and
wherein the plurality of first unit pixels are arranged in the plurality of biometric information measurement areas in a matrix pattern in the first direction and the second direction.

17. The display device of claim 15, wherein the plurality of biometric information measurement areas each comprise:
a plurality of first unit pixels each comprising a plurality of display pixels and a light-sensing pixel; and
a plurality of second unit pixels each comprising a plurality of display pixels and an infrared light-emitting pixel,
wherein the plurality of first unit pixels and the plurality of second unit pixels are arranged alternately in the first direction and the second direction such that they are arranged in a matrix pattern in the plurality of biometric information measurement areas.

18. A display device comprising:
a display panel having a display area divided into a plurality of image display areas and a plurality of biometric information measurement areas;
a plurality of display pixels arranged in the plurality of image display areas and the plurality of biometric information measurement areas;
a plurality of light-sensing pixels arranged in the plurality of biometric information measurement areas;
a plurality of infrared light-emitting pixels arranged in the plurality of biometric information measurement areas;
a display scan driver configured to drive the plurality of display pixels and the plurality of infrared light-emitting pixels to emit light;
a light-sensing scan driver configured to drive the plurality of light-sensing pixels to sense light; and
a main driver circuit configured to receive light-sensing signals from the plurality of light-sensing pixels,
wherein a first biometric information measurement area among the plurality of biometric information measurement areas is extended in a length direction of the display area between a first image display area and a central image display area, which are extended in a length direction of the display panel, and
wherein a second biometric information measurement area among the plurality of biometric information measurement areas is extended in the length direction of the display area between the central image display area and a second image display area, which are extended in the length direction of the display panel,
wherein the first image display area and the second image display area are disposed on opposite sides of the central image display area.

19. The display device of claim 18, wherein the first image display area, the first biometric information measurement area, the central image display area, the second biometric information measurement area, and the second image display area are formed in the display area such that they are parallel to one another in a width direction of the display panel and extended in the length direction of the display area.

20. The display device of claim 18, wherein the plurality of light-sensing pixels and the plurality of infrared light-emitting pixels are entirely disposed outside of the central image display area.

21. An electronic device including a display device, wherein the display device comprising:
a display panel having a display area divided into a plurality of image display areas and a plurality of biometric information measurement areas;
a plurality of display pixels arranged in the plurality of image display areas and the plurality of biometric information measurement areas;
a plurality of light-sensing pixels arranged in the plurality of biometric information measurement areas;
a plurality of infrared light-emitting pixels arranged in the plurality of biometric information measurement areas;
a display scan driver configured to drive the plurality of display pixels and the plurality of infrared light-emitting pixels to emit light;
a light-sensing scan driver configured to drive the plurality of light-sensing pixels to sense light; and
a main driver circuit configured to detect a pulse wave signal and measure biometric information using light-sensing signals received from the plurality of light-sensing pixels,
wherein the plurality of biometric information measurement areas are disposed in parallel rectangular shapes.
